(19) United States
(12) Patent Application Publication
(10) Pub. No.: US 2019/0194605 A1
(43) Pub. Date: Jun. 27, 2019

(54) IN-VITRO METHOD FOR IDENTIFYING AND ANALYZING SECRETION PROTEINS USING A THREE-DIMENSIONAL CELL CULTURE MODEL OF THE SWEAT GLAND

(71) Applicant: Henkel AG & Co. KGaA, Duesseldorf (DE)

(72) Inventors: Patricia Klaka, Leverkusen (DE); Bernhard Banowski, Duesseldorf (DE); Sabine Gruedl, Erkelenz (DE); Thomas Welss, Duesseldorf (DE); Melanie Giesen, Geldern (DE)

(73) Assignee: Henkel AG & Co. KGaA, Duesseldorf (DE)

(21) Appl. No.: 16/331,482

(22) PCT Filed: Aug. 3, 2017
(86) PCT No.: PCT/EP2017/069653

§ 371 Date: Mar. 7, 2019

(30) Foreign Application Priority Data
Sep. 9, 2016 (DE) 10 2016 217 174.7

(51) Int. Cl. C12N 5/00

(52) U.S. Cl. CPC C12N 5/0062; C12N 2533/72; C12N 2533/54; C12N 5/0018

(57) ABSTRACT

The present disclosure relates to an in-vitro method for identifying and analyzing secretion proteins, in which a three-dimensional sweat gland equivalent having from about 500 to about 500,000 sweat gland cells and a diameter of from about 100 to about 6,000 μm is firstly provided and then any secretion proteins present in this equivalent are infected and analyzed. In a further method step c) the influence of test substances on the proteins identified previously in step b) is examined. Since the three-dimensional sweat gland equivalents used in step a) comprise differently differentiated cells and portray the in-vivo situation well, the measurement data obtained with the in-vitro method as contemplated herein can be transferred well to the in-vivo situation.

### CROSS-REFERENCE TO RELATED APPLICATION

This application is a U.S. National-Stage entry under 35 U.S.C. § 371 based on International Application No. PCT/EP2017/069653, filed Aug. 3, 2017, which was published under PCT Article 21(2) and which claims priority to German Application No. 10 2016 217 174.7, filed Sep. 9, 2016, which are all hereby incorporated in their entirety by reference.

### TECHNICAL FIELD

The present disclosure relates to an in-vitro method for identifying and analyzing secretion proteins, in which a three-dimensional sweat gland equivalent having from about 500 to about 500,000 sweat gland cells and a diameter of from about 100 to about 6,000 μm is firstly provided and then any secretion proteins present in this equivalent are identified and analyzed. The three-dimensional sweat gland equivalents used as contemplated herein comprise ordered structures and differently differentiated cells and show a capability of reaction both at gene expression level and at protein expression level to an external stimulus, for example a cholinergic stimulus by acetylcholine (also referred to as ACh).

### BACKGROUND

The washing, cleaning, and care of one's own body is a basic human need, and modern industry is continually attempting to meet these human needs in a variety of ways. Especially important for daily hygiene is the lasting elimination or at least reduction of body odor and armpit moisture. Underarm wetness and body odor are caused by the secretion from eccrine and apocrine sweat glands in the human axillae. Whilst the eccrine glands are used to regulate body temperature and are responsible for the creation of underarm wetness, the apocrine glands release a viscous secretion in response to stress, which leads to unpleasant body odor as a result of bacterial decomposition.

Initial research on natural eccrine and apocrine sweat glands was carried out already at the start of the 20th century in order to classify these skin appendages belonging to the group of exocrine glands. On this basis, sweat glands can be divided into apocrine and eccrine sweat glands and a mixed form formed of apocrine and eccrine sweat glands (also referred to as apocrine sweat glands). The aforementioned forms can be differentiated on the basis of morphological and characteristic features.

The eccrine sweat gland, for example the human eccrine sweat gland, belongs to the unbranched spiralled tubular glands and can be divided into the secretory end part (also referred to as the coil), the dermal excretory duct (also referred to as the duct) and the epidermal excretory duct (also referred to as the acrosyringium). The cells provided in these gland portions have different tasks and functions, such as secretion in the coil, reabsorption of ions in the duct, and delivery of the secretion, for example the sweat, to the surrounding skin by the acrosyringium. The eccrine sweat glands are stimulated primarily by the neurotransmitter acetylcholine (ACh), however a purinergic stimulation (for example with ATP/UTP) and an αβ-adrenergic stimulation (for example with noradrenaline) is possible.

In view of the avoidance of underarm wetness and/or body odor, it is therefore desirable to reduce and/or prevent the secretion of eccrine and/or apocrine sweat glands. This can be achieved for example by blocking the excretory ducts of the eccrine sweat glands by what are known as plugs. To this end, antiperspirant aluminium and/or aluminium-zirconium salts are used in the prior art, however consumers are sceptical towards them. Furthermore, antibacterial agents that prevent the bacterial breakdown of sweat are used in the prior art. Agents of this kind, however, may negatively influence the natural microflora of the skin under the axillae. It is therefore desirable to provide cosmetic agents that are able to reliably prevent underarm wetness and/or body odor and which are free from aluminium and/or aluminium-zirconium salts and compounds having an antibacterial effect. One possibility for providing such agents lies in the use of substances which effectively prevent the stimulation and/or the biological processes of the sweat glands and thus reduce or prevent the secretion of sweat. In order to be able to identify substances of this kind, in-vivo tests can be performed with test participants. Such tests, however, are complex and do not permit screening methods with high throughput rates. On the other hand, it is possible to use in-vitro tests employing cell models of sweat glands on which the influence of test substances on the stimulation of the sweat glands can be examined.

In order to make it possible for the test results obtained in-vivo to be transferred effectively to the in-vivo situation, the used cell model of the sweat gland must emulate the in-vivo situation as accurately as possible. To this end, three-dimensional cell models are necessary, since the two-dimensional models known in the prior art are not sufficiently physiologically close to the native sweat gland and therefore reflect the in-vivo situation only insufficiently. In addition, it is necessary to explain the sweat secretion mechanism. This is because only in this way can “biological targets”, for example proteins produced by the sweat gland cells, be identified, the influencing of which by the test substances leads to a reduced sweat production. A possible biological target which could be related to sweat production is constituted by secretion proteins, which are formed by the sweat gland cells during sweat secretion.

There is thus also a need for in-vitro methods with the aid of which biological targets which are responsible for increased sweat production can be identified and analyzed. Following the identification and analysis of targets of this kind, the influence of various test substances on these targets is examined. In-vitro methods of this kind should be suitable for standardization and should be executable economically and quickly, such that it is made possible to determine the influence of test substances on the biological targets in screening methods with high throughput rates.

### BRIEF SUMMARY

The object of the present disclosure was therefore to provide an in-vitro method for identifying and analyzing secretion proteins, which method can be standardized and can be executed economically and quickly, and the results of which can be transferred to the in-vivo situation.

It has now surprisingly been found that it has been made possible to identify and analyze secretion proteins with the aid of specific three-dimensional sweat gland equivalents. The used three-dimensional sweat gland equivalents have an ordered structure. Furthermore, the primary sweat gland cells of these equivalents develop the same characteristics as natural sweat glands. Thus, the measurement data with regard to the identification and analysis of secretion proteins obtained with these equivalents can be transferred very well to the in-vivo situation.

A first subject of the present disclosure is thus an in-vitro method for identifying and analyzing secretion proteins in the human sweat gland, said method comprising the following method steps:

a) providing at least one three-dimensional sweat gland equivalent, comprising from about 500 to about 500,000 sweat gland cells, wherein the at least one three-dimensional sweat gland equivalent has a diameter of from about 100 to about 6,000 μm, and
b) identifying and analyzing at least one secretion protein in the at least one three-dimensional sweat gland equivalent provided in method step a).

The three-dimensional sweat gland equivalents used in the method as contemplated herein form an ordered structure and comprise differentiated cells with the same characteristics as natural sweat glands. Furthermore, these equivalents show a response at gene expression level and at protein expression level to a stimulus by acetylcholine (ACh). The results obtained with the method as contemplated herein thus can be transferred well to the in-vivo situation. By employing the use of cultivated, primary sweat gland cells in the production of the equivalents, a high standardization can be achieved, since a large number of equivalents having the same property can be produced from the cultivated cells. Furthermore, by employing the use of cultivated primary sweat gland cells, equivalents having approximately the same numbers of sweat gland cells can be produced, which likewise ensures a high capability of standardization.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

The term “secretion proteins” shall be understood as contemplated herein to mean proteins that are formed by the cells of the three-dimensional sweat gland equivalents and are secreted in the extracellular space. Proteins of this kind are also referred to in the literature as secretory proteins.

Furthermore, a three-dimensional sweat gland equivalent is understood as contemplated herein to mean a cell model formed from sweat gland cells which has an extent in all three spatial directions and in which the cells show a function similar, for example a function identical, that that of the cells of a natural sweat gland.

In method step a) of the method as contemplated herein at least one three-dimensional sweat gland equivalent having a specific cell number and a specific diameter is firstly provided.

Particularly preferred three-dimensional sweat gland equivalents have specific diameters. It is therefore advantageous as contemplated herein if the at least one three-dimensional sweat gland equivalent provided in method step a) has a diameter of from about 100 to about 4,000 μm, of from about 100 to about 2,000 μm, for example of from about 200 to about 1,500 μm. The diameter of the spherical sweat gland equivalents used as contemplated herein can be specified for example by employing microscopic measurement using the “CellSens” software.

Within the scope of the present disclosure it is preferred if the sweat gland equivalents used in method step a) are free from matrix compounds and/or carriers. Matrix compounds shall be understood here to be compounds which are capable of forming spatial networks. This does not include, however, the substances which are produced and excreted by the cells of the equivalents themselves and are capable of forming spatial networks. Furthermore, “carriers” in the sense of the present disclosure are understood to mean self-supporting substances which can be used as a substrate or framework for the sweat gland cells. In accordance with a preferred embodiment of the present disclosure the at least one three-dimensional sweat gland equivalent provided in method step a) is free from matrix compounds and/or carriers, for example free from matrix compounds and carriers.

The term “free from” as contemplated herein is understood to mean that the three-dimensional sweat gland equivalents contain less than about 1% by weight of matrix compounds and/or carriers in relation to the total weight of the three-dimensional sweat gland equivalent. It is therefore advantageous within the scope of the present disclosure if the three-dimensional sweat gland equivalents used in method step a) contain 0 to about 1% by weight, 0 to about 0.5% by weight, 0 to about 0.2% by weight, for example 0% by weight of matrix compounds and carriers, in each case in relation to the total weight of the three-dimensional sweat gland equivalent.

In this regard it is particularly advantageous if the three-dimensional sweat gland equivalents used in method step a) are free from specific matrix compounds and carriers. It is therefore preferred if the three-dimensional sweat gland equivalent does not contain any matrix compounds and/or carriers which are selected from the group of collagens, for example type I and/or type III and/or type IV collagen, scleroproteins, gelatines, chitosans, glucosamines, glycosaminoglycans (GAGs), heparin sulfate proteoglycans, sulfated glycoproteins, growth factors, crosslinked polysaccharides, crosslinked polypeptides, and mixtures thereof.

The three-dimensional sweat gland equivalent provided in method step a) is particularly an equivalent of the eccrine and/or apocrine human sweat gland. Preferred embodiments of the present disclosure are therefore exemplified in that the at least one three-dimensional sweat gland equivalent provided in method step a) is a three-dimensional sweat gland equivalent of the eccrine and/or apocrine human sweat gland. Sweat gland equivalents of this kind are particularly well suited for identifying and analyzing secretion proteins and for determining the influence of test substances on these proteins.

It is additionally particularly preferred as contemplated herein if the three-dimensional sweat gland equivalent provided in method step a) has been produced from human eccrine and/or apocrine sweat glands. It is therefore advantageous within the scope of the present disclosure if the at least one three-dimensional sweat gland equivalent provided in method step a) is a three-dimensional sweat gland equivalent obtained from natural human eccrine and/or apocrine sweat gland cells.

It has additionally been found to be advantageous as contemplated herein if the three-dimensional sweat gland equivalents provided in method step a) comprise at least one specific cell type. The use of equivalents of this kind leads to a particularly good identification and analysis of secretion proteins. Preferred embodiments of the present disclosure are therefore exemplified in that the at least one three-dimensional sweat gland equivalent provided in method step a) contains at least one cell type, selected from the group of (i) coil cells, for example clear cells, dark cells, and myoepithelial cells, (ii) duct cells, and (iii) mixtures thereof. The term “clear cells” shall be understood as contemplated herein to mean cells which have a clear or uncoloured cytoplasm when stained with dyes, for example with haematoxylin and eosin. “Clear cells” of this kind are secretory cells of the epithelium, wherein the plasma membrane is heavily folded at the apical and lateral surface. The cytoplasm of these “clear cells” contains high amounts of glycogen and many mitochondria. The cells are arranged in contact with the lumen. The aqueous component of sweat, which contains electrolytes and inorganic substances, is excreted by this cell type. By contrast, the aforementioned “dark cells” are cells of which the vacuoles have a positive acid mucopolysaccharide staining, the cytoplasm of which cells thus can be stained by dyes. These “dark cells” are in contact with the basal membrane and comprise only mitochondria in comparison to the “clear cells”. Macromolecules, such as glycoproteins, are separated from these “dark cells”. The aforementioned “myoepithelial cells” are understood to mean contractile epithelial cells which have a cytoskeleton with what are known as gap junctions and can therefore contract. In this way, the delivery of secretion from the gland end portions is supported. Cells of this kind are situated between the basal membrane and the aforementioned “clear cells” and “dark cells”. Lastly, the term “duct cells” as contemplated herein are understood to mean cells which form the wall of the duct and have a stratified cubic epithelium. The aforementioned cell types can be determined, besides by the use of haematoxylin and eosin, also by employing immunocytochemical colourings with use of markers specific for these cells. A specific marker that can be used for myoepithelial cells is alpha-smooth muscle actin also referred to as α-SMA). “Clear cells”, for example Substance P and S100, are suitable as specific markers. Furthermore, the markers known by the name CGRP (calcitonin-gene related peptide) can be used for “dark cells”, and the specific markers Cytokeratin 10 (also referred to as CK10) and CD200 can be used for duct cells.

Particularly preferred three-dimensional sweat gland equivalents used in method step a) will be described hereinafter.

An exemplary embodiment of this subject of the present disclosure is therefore the provision of a three-dimensional sweat gland equivalent of the human eccrine and/or apocrine sweat gland, comprising from about 500 to about 500,000 sweat gland cells, wherein the three-dimensional sweat gland equivalent has a diameter of from about 200 to about 1,500 μm.

An exemplary embodiment of this subject of the present disclosure is furthermore the provision of a three-dimensional sweat gland equivalent obtained from natural human eccrine and/or apocrine sweat gland cells, comprising from about 500 to about 500,000 sweat gland cells, wherein the three-dimensional sweat gland equivalent has a diameter of from about 200 to about 1,500 μm.

In addition, an exemplary embodiment of this subject of the present disclosure is the provision of a three-dimensional sweat gland equivalent, comprising from about 500 to about 500,000 sweat gland cells, wherein the three-dimensional sweat gland equivalent has a diameter of from about 200 to about 1,500 μm and contains at least one cell type, selected from the group of clear cells, dark cells, myoepithelium cells, duct cells, and mixtures thereof.

In addition, an exemplary embodiment of this subject of the present disclosure is the provision of a three-dimensional sweat gland equivalent obtained from natural human eccrine and/or apocrine sweat gland cells, comprising from about 500 to about 500,000 sweat gland cells, wherein the three-dimensional sweat gland equivalent has a diameter of from about 200 to about 1,500 μm and contains at least one cell type, selected from the group of clear cells, dark cells, myoepithelium cells, duct cells, and mixtures thereof.

Furthermore, an exemplary embodiment of this subject of the present disclosure is the provision of a three-dimensional sweat gland equivalent of the human eccrine and/or apocrine sweat gland, comprising from about 500 to about 500,000 sweat gland cells, wherein the three-dimensional sweat gland equivalent has a diameter of from about 200 to about 1,500 μm and contains 0% by weight of matrix compounds and carriers, in relation to the total weight of the three-dimensional sweat gland equivalent.

In addition, an exemplary embodiment of this subject of the present disclosure is the provision of a three-dimensional sweat gland equivalent of the human eccrine and/or apocrine human sweat gland, comprising from about 500 to about 500,000 sweat gland cells, wherein the three-dimensional sweat gland equivalent has a diameter of from about 200 to about 1,500 μm and contains 0% by weight of matrix compounds and carriers in relation to the total weight of the three-dimensional sweat gland equivalent, wherein the matrix compounds and/or carriers are selected from the group of collagens, for example type I and/or type III and/or type IV collagen, scleroproteins, gelatines, chitosans, glucosamines, glycosaminoglycans (GAGs), heparin sulfate proteoglycans, sulfated glycoproteins, growth factors, crosslinked polysaccharides, crosslinked polypeptides, and mixtures thereof.

The three-dimensional sweat gland equivalents provided in method step a) have a higher capability for standardization and a higher availability than isolated sweat glands and are closer to the in-vivo situation than one-dimensional and two-dimensional sweat gland models. Furthermore, these equivalents represent an economical alternative to in-vivo studies on humans, since, by employing these equivalents secretion proteins can be identified and the influence thereof on sweat secretion can be analyzed. This is because the three-dimensional sweat gland equivalents simulate the sweat gland in-vivo both in respect of their structure and in respect of their histological composition, and therefore the information obtained with these equivalents can be transferred well to the human model.

The three-dimensional sweat gland equivalent provided in method step a) can be obtained for example by the following production method.

In a first step isolated sweat glands are firstly provided which can be obtained from skin biopsies or the like and which have been removed from their natural environment. The isolated sweat glands of the first step are obtained by the isolation of natural sweat glands, for example natural eccrine and/or apocrine sweat glands, from the human skin, wherein the native sweat glands are isolated by enzymatic digestion of the human skin with use of a mixture of from about 2 to about 3 mg/ml collagenase II and from about 0.1 to about 0.2 mg/ml thermolysin for from about 3 to about 6 hours at from about 35 to about 40° C., for example at about 37° C.

These isolated sweat glands in a second step are cultivated in a specific culture medium in order to obtain a cell culture. A particularly good cultivation of the isolated sweat gland cells obtained in the first step is achieved if a mixture of DMEM and Ham's F12 in a weight ratio of about 3:1, which additionally contains about 10% by weight of foetal calf serum (FCS) in relation to the total weight of the mixture, is used as culture medium. These cells are cultivated in the above-described culture medium for from about 7 to about 28 days, for example for about 14 days, at a temperature of from about 36 to about 38° C. and a CO₂ content of about 5% by weight, in relation to the total weight of the atmosphere used for cultivation.

In the third step a cell preparation of primary sweat gland cells is produced from the cultivated cells in a culture medium, wherein the cell count of the primary sweat gland cells in the cell preparation is from about 50 to about 250,00 cells per μL, preferentially from about 100 to about 10,000 cells per μL, from about 150 to about 5,000 cells per μL, more from about 200 to about 3,200 cells per μL, even more from about 300 to about 1,000 cells per μL, for example from about 400 to about 600 cells per μL of culture medium. The cell preparation of primary sweat gland cells is produced by detaching the sweat gland cells cultivated in the second step, for example by gentle trypsinisation, cultivating these detached sweat gland cells in monolayer cultures, suspending the cultivated primary sweat gland cells in a culture medium, and adjusting the cell count. For the cultivation of the detached sweat gland cells and for production of the cell suspension it has proven to be advantageous if a mixture of DMEM and Ham's F12 in a weight ratio of about 3:1, which additionally contains about 10% by weight of foetal calf serum (FCS) in relation to the total weight of the mixture, is used as culture medium. The cultivation of the detached sweat gland cells is performed at a temperature of from about 36 to about 38° C. and a CO₂ content of about 5% by weight in relation to the total weight of the atmosphere used for cultivation, to confluency.

In a fourth step, from about 10 to about 100 100 μL, preferentially from about 20 to about 80 μL, from about 30 to about 70 μL, for example from about 40 to about 60 μL of this cell preparation are then cultivated in a hanging state, that is to say in the form of a droplet hanging down from a surface in a freely floating manner, until the three-dimensional sweat gland equivalents have formed. In this regard the use of what are known as hanging drop wells, as disclosed for example in the laid-open application WO 2012/014047 A1 and available commercially from the company Insphero as GravityPLUS® sowing plate with SureDrop® Inlet delivery system and GravityTRAP® harvesting plate, has proven to be advantageous. The cell preparation is cultivated in the hanging state for a period of from about 1 to about 25 days, for example from about 2 to about 7 days, at a temperature of from about 36 to about 38° C. and a CO₂ content of about 5% by weight, in relation to the total weight of the atmosphere used for cultivation. Here, it is preferred if, during the cultivation period, for example after from about 1 to about 3 days, about 40 volume percent, in relation to the total volume of the aforementioned cell preparation, of the culture medium of the cell preparation have been replaced by fresh culture medium.

After isolation of the obtained equivalents by addition of from about 50 to about 200 μL, for example from about 70 to about 100 μL, of culture medium, the equivalents can be used directly for method step b) of the method as contemplated herein or can be newly cultivated. The obtained equivalents are freshly cultivated for a period of from about 1 to about 6 days at a temperature of from about 36 to about 38° C. and a CO₂ content of about 5% by weight, in relation to the total weight of the atmosphere used for cultivation.

The three-dimensional sweat gland equivalents are therefore particularly provided in method step a) by employing the method described hereinafter, which comprises the following steps in the stated order:

(i) providing isolated sweat glands, wherein the isolated sweat glands are obtained by isolation of natural eccrine and/or apocrine sweat glands from the human skin and subsequent suspension of these isolated sweat glands in culture medium,
(ii) providing a cell preparation of primary sweat gland cells from the sweat glands isolated in method step (i), wherein the cell count of the primary sweat gland cells in the cell preparation is from about 400 to about 600 cells per μL, and wherein the cell preparation of primary sweat gland cells has a volume of from about 40 to about 60 μL,
(iii) cultivating the cell preparation provided in method step (ii) in a hanging state, wherein the hanging state of the cell preparation is achieved by using a hanging drop multiwell plate, and wherein during the cultivation period about 40 volume percent, in relation to the total volume of the cell preparation used in this method step, of the culture medium of the cell preparation are replaced by fresh culture medium,
(iv) isolating the three-dimensional sweat gland equivalent obtained in method step (iii), wherein the isolation of the three-dimensional sweat gland equivalent is achieved by adding from about 50 to about 200 μL of culture medium to detach the model,
(v) optionally cultivating the three-dimensional sweat gland equivalent isolated in method step (iv) for a period of from about 1 to about 6 days at a temperature of from about 36 to about 38° C. and a CO₂ content of about 5% by weight, in relation to the total weight of the atmosphere used for cultivation.

Since, within the scope of the present disclosure, secretion proteins of the human eccrine and/or apocrine sweat gland are to be identified and analyzed, the equivalents provided in step a) are produced with use of natural human eccrine and/or apocrine sweat glands. Natural eccrine and/or apocrine sweat glands are understood here to mean eccrine and/or apocrine sweat glands which have been isolated from human skin, for example from human skin biopsies or by other methods.

Furthermore, the three-dimensional sweat gland equivalents provided in step a) are produced exclusively with use of in-vitro methods. Consequently, no method steps are contained in which in-vivo methods are used. These equivalents can thus also be used to test substances which are intended for cosmetic use. Furthermore, this production method allows economical production of standardized equivalents which can be used in screening methods with high throughput rates. In addition this production method results in three-dimensional equivalents which form ordered structures, comprise differently differentiated structures, and express sweat gland-specific markers, such that good transferability of in-vitro data to the in-vivo situation is made possible.

A production method for the three-dimensional sweat gland equivalents provided in method step a) of the method as contemplated herein is disclosed for example in German application DE 10 2015 222 279, with reference being made hereby to the full content of that document.

In the second method step of the method as contemplated herein at least secretion protein in the three-dimensional sweat gland equivalent provided in method step a) is analyzed.

Preferred biological targets as contemplated herein are secretion proteins with antimicrobial effect. It is therefore preferred if the at least one secretion protein in method step b) is an antibacterial secretion protein.

Within the scope of the present disclosure specific secretion proteins, for example specific secretion proteins with antibacterial effect, are advantageously identified and analyzed. Preferred embodiments of the present disclosure are therefore exemplified in that the at least one secretion protein, for example the at least one antibacterial secretion protein, in method step b) is selected from the group of mucins, dermicidins, beta-defensins, secretoglobins, lysozyme, albumin, lactate dehydrogenase (LDH), galanin, prolactin-induced protein (PIP), vasoactive intestinal peptide (VIP), zinc-alpha-2-glycoprotein (ZA2G), lipophilin, apolipoproteins, S100 proteins, calcium-dependent activator protein for secretion (CADPS), substance P, calcitonin-gene related peptide (CGRP) and mixtures thereof. As contemplated herein, mucins are proteins that belong to the proteoglycans and therefore contain approximately 40 to about 80% by weight of carbohydrates, in relation to the total weight of the mucin. Furthermore, their protein fraction of up to about 50% by weight, in relation to the total amount of protein, of the amino acids serine and threonine and/or proline. They additionally have a high molecular weight and a high negative charge by sialyl or sulfate groups. As a result of this anionic character and the presence of hydroxy groups within the mucins, these are capable of binding a high amount of water and forming gel-like networks. Dermicidins, which are also referred to as proteolysis-inducting factor (PIF), are proteins that have an antimicrobial effect and that are secreted from human eccrine sweat glands. By breaking down these proteins with the aid of proteases, the antimicrobial effect can be further increased. Beta-defensins are understood to be cationic proteins with a mean molecular weight of from about 2 to about 6 kDa, which have an antimicrobial effect in respect of gram-negative and gram-positive bacteria, fungi and viruses. These proteins have three intramolecular disulphide bridges and are divided on the basis of their size and pattern of their disulphide bonds into alpha-, beta- and theta-defensins. Secretoglobins are small protein dimers that are connected to one another via disulphide bridges and occur exclusively in mammals. For example, uteroglobin, uteroglobin-related protein 1, uteroglobin-related peptide 2, mammaglobin-A and mammaglobin-B belong to the family of secretoglobins. Apolipoproteins refer to the protein fraction of lipoproteins, for example chylomicrons, VLDL, LDL, IDL and HDL, which transports the water-insoluble lipids in blood. S100 proteins are calcium-binding proteins with a molecular mass of from about 8 to about 14 kDa which influence a large number of cellular processes.

The aforementioned proteins can be secreted by the sweat gland cells of the three-dimensional sweat gland equivalents, wherein their secretion is dependent on the sweat production. Thus, these proteins are particularly suitable as biological targets for examination of the secretion mechanism.

The identification and analysis of the secretion protein, for example the aforementioned proteins, is performed as contemplated herein with use of specific methods. It is therefore preferred as contemplated herein if the identification and analysis in method step b) are performed by employing methods selected from the group of molecular biological methods, protein analyzes, assays for determining functionality, and combinations thereof. Molecular biological methods that can be used within the scope of the present disclosure are, for example, NGS (next generation sequencing) analysis and qRT-PCR (quantitative real-time PCR). By employing these methods the aforementioned proteins can be identified by employing gene expression analyzes and quantitatively determined. The expression level of the proteins obtained in the three-dimensional sweat glad equivalents were compared with the expression level of these proteins in human sweat gland samples and in full skin samples. The expression level of these proteins in the three-dimensional sweat gland equivalents and in the human sweat gland was significantly higher than in the full skin samples, and therefore these proteins can represent specific marker proteins for the sweat gland. Furthermore, the obtained expression of these proteins in the three-dimensional sweat gland equivalents was comparable to the expression of these proteins in the human sweat gland. The sweat gland equivalents used in the method as contemplated herein therefore emulate the in-vivo situation outstandingly and thus ensure a good transferability of the in-vitro results to the in-vivo situation.

Suitable protein analyzes are, for example, immunolabellings of the aforementioned proteins by employing specific markers, such as the methods of immunofluorescence, Western Blot analysis, and/or ELISA. A quantitative determination of the aforementioned proteins is likewise possible with the two last-mentioned methods.

Within the scope of the method as contemplated herein it has proven to be advantageous if, after method step b), a further method step c) is performed. In this method step c) the influence of various test substances on the secretion proteins identified in method step b), for example the aforementioned specific proteins, is determined. Preferred embodiments of the present disclosure are therefore exemplified in that, in an additional method step c), the influence of compounds on the secretion proteins identified in method step b) is examined. The compounds used in method step c) are inhibitors of these proteins, if the proteins increase the sweat secretion. If, however, the aforementioned proteins reduce the sweat secretion, activators are used as compounds in method step c).

In this regard it is preferred if, in method step c), specific methods are used for determining the influence of the compound on the proteins identified in method step b). It is therefore advantageous as contemplated herein if the influence of the at least one compound in method step c) is provided by employing methods selected from the group of molecular biological methods, protein analyzes, assays for determining functionality, and combinations thereof. With regard to the methods, reference is made to the methods mentioned above and used in method step b), wherein these can be used equally to carry out method step c).

The following examples shall explain the present disclosure, but are not intended to be limiting.

### Examples

### 1 Provision of the Three-Dimensional Sweat Gland Equivalents (Method Step a))

### 1.1 Isolation of the Sweat Glands

The natural sweat glands were obtained from human tissue samples, or what are known as biopsies, which originated from plastic surgery operations performed on patients who had consented to the use of the material for research purposes. The used tissue was removed during the course of upper arm lift and face lift procedures. The eccrine and apocrine sweat glands from the axilla region were isolated herefrom.

To this end, the biopsy in question was divided into small pieces and was then cut into pieces measuring at most approximately 1 cm×1 cm. The skin was then digested with a mixture of equal parts of collagenase II (5 mg/ml) and thermolysin (0.25 mg/ml) at 37° C. in an incubator for approximately 3.5 to 5 hours, until the connective tissue was almost fully digested. This mixture was then centrifuged at 1200 rpm for 5 minutes and the supernatant was discarded so as to remove the enzyme solution and the excess fat. The resultant pellet was taken up in DMEM solution and the solution was transferred to a Petri dish. Intact sweat glands were isolated under a binocular on the basis of a microcapillary and were transferred into fresh DMEM medium.

1.2 the Isolated Natural Sweat Glands were Cultivated.

The sweat glands isolated in step 1.1 were placed in culture flasks coated with collagen I, and then 25 ml culture medium were added. After cultivation for 7 to 21 days in an incubator at 37° C. and 5% CO₂ the washed-out sweat gland cells were detached and cultivated again in culture flasks coated with collagen I to confluency (monolayer culture of the primary sweat gland cells).

The composition of the used culture medium was as follows:

| Constituents of the medium | |
|---|---|
| DMEM/Ham's F12 Nutrient Mix | 3:1 |
| Foetal calf serum (FCS) | 10% |
| EGF | 10 ng/ml |
| Hydrocortisone | 0.4 μg/ml |
| Insulin | 0.12 UI/ml |
| Cholera toxin | 10⁻¹⁰M |
| Adenine | 2.43 g/ml |
| Gentamicin | 25 μg/ml |
| Penicillin G | 100 UI/ml |
| Triiodothyronine | 2 * 10⁻⁹M |
| Ascorbyl-2-phosphate | 1 mM |

### 1.3 Production of the Cell Preparation and the Three-Dimensional Sweat Gland Equivalent

Once the exact cell counts of the above monolayer cultures of the primary sweat gland cells had been determined, these were set to a cell count of 10 to 5,000 cells per μl with use of the above culture medium, and then this cell suspension was transferred to wells of a GravityPLUS® sowing plate by employing the SureDrop® Inlet delivery system (both from the company Inshpero AG, Switzerland), with 50 μl of the cell suspension being introduced into each well. The cultivation was performed at 36 to 38° C. and a CO₂ content of 5% by weight, in relation to the total weight of the atmosphere used for cultivation. After 1 to 3 days 40 vol. % of the medium in the wells of the GravityPLUS® sowing plate were replaced by fresh culture medium. After 2 to 7 days cultivation the 3D sweat gland equivalents were harvested by adding 50 to 200 μL of culture medium and were transferred into a GravityTRAP® plate (company Insphero AG, Switzerland). Prior to harvesting, the GravityTRAP® plate was wetted with 60 to 100 μL keratinocyte medium with the aid of a multi-duct pipette in order to minimise the formation of air bubbles and prevent the loss of the three-dimensional sweat gland equivalents. After harvesting the plate was harvested for 1 to 5 minutes at 100 to 300×g in order to remove air bubbles. Part of the three-dimensional sweat gland equivalents was analyzed, whereas a further part was cultivated for 1 to 6 days in the depressions of the harvesting plate at 37° C. and 5% by weight CO₂, in relation to the total weight of the atmosphere used for cultivation.

### 2. Identification and Analysis of a Secretion Protein (Method Step b))

The gene expression of the aforementioned particularly preferred secretion proteins in the three-dimensional sweat gland equivalents was able to be detected by employing NGS analyzes. To this end, the RNA-Seq method (also referred to as “total transcription shotgun sequencing”) of the company Illumina was used. In this method the RNA is converted into cDNA and then the method of DNA sequencing is applied. For this purpose the mRNA is firstly separated from the rRNA, with use of what are known as “ribosomal depletion kits” and then fragmented, wherein the fragmentation can occur both before and after the conversion into cDNA. The sequencing is then performed, wherein the incorporation of an individual nucleotide into the cDNA is converted into an electrical signal. This sequencing can be performed for example by employing the Illumina genome Analyzer, with execution of the following steps: cDNA fragmentation, purification, repair of the fragment ends, ligation of the adapters to the sample, separation of the samples with an agarose gel on the basis of their size, PCR and purification and sequencing. After the sequencing the read mapping is performed, wherein the read fragments (also referred to as reads) are associated with the reference genome. In addition, substance P, S100 proteins, and calcitonin-gene related peptide (CGRP) could also be detected with the aid of immunofluorescence dyes.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. An in-vitro method for identifying and analyzing secretion proteins in the human sweat gland, said method comprising the following method steps:
a) providing at least one three-dimensional sweat gland equivalent, comprising from about 500 to about 500,000 sweat gland cells, wherein the three-dimensional sweat gland equivalent has a diameter of from about 100 to about 6,000 μm, and
b) identifying and analyzing at least one secretion protein in the at least one three-dimensional sweat gland equivalent provided in method step a).

2. The method according to claim 1, wherein the at least one three-dimensional sweat gland equivalent provided in method step a) has a diameter of from about 100 to about 4,000 μm.

3. The method according to claim 1, wherein the at least one three-dimensional sweat gland equivalent provided in method step a) is free from matrix compounds and/or carriers.

4. The method according to claim 3, wherein the matrix compounds and/or carriers are selected from the group of collagens, scleroproteins, gelatines, chitosans, glucosamines, glycosaminoglycans (GAGs), heparin sulfate proteoglycans, sulfated glycoproteins, growth factors, crosslinked polysaccharides, crosslinked polypeptides, and mixtures thereof.

5. The method according to claim 1, wherein the at least one three-dimensional sweat gland equivalent provided in method step a) comprises at least one cell type, selected from the group of (i) coil cells, (ii) duct cells, and (iii) mixtures thereof.

6. The method according to claim 1, wherein the at least one secretion protein in method step b) is an antibacterial secretion protein.

7. The method according to claim 1, wherein the at least one secretion protein, in particular the at least one antibacterial secretion protein in method step b) is selected from the group of mucins, dermicidins, beta-defensins, secretoglobins, lysozyme, albumin, lactate dehydrogenase (LDH), galanin, prolactin-induced protein (PIP), vasoactive intestinal peptide (VIP), zinc-alpha-2-glycoprotein (ZA2G), lipophilin, apolipoproteins, S100 proteins, calcium-dependent activator protein for secretion (CADPS), substance P, calcitonin-gene related peptide (CGRP) and mixtures thereof.

8. The method according to claim 1, wherein the identification and analysis in method step b) are performed by means of methods selected from the group of molecular biological methods, protein analyses, assays for determining functionality, and combinations thereof.

9. The method according to claim 1, wherein, in an additional method step c), the influence of at least one compound on the at least secretion protein identified in method step b) is examined.

10. The method according to claim 9, wherein the influence of the at least one compound is examined in method step c) by means of methods selected from the group of molecular biological methods, protein analyses, assays for determining functionality, and combinations thereof.

11. The method according to claim 1, wherein the at least one three-dimensional sweat gland equivalent provided in method step a) has a diameter of from about 100 to about 2,000 μm.

12. The method according to claim 1, wherein the at least one three-dimensional sweat gland equivalent provided in method step a) has a diameter of from about 200 to about 1,500 μm.

13. The method according to claim 1, wherein the at least one three-dimensional sweat gland equivalent provided in method step a) is free from matrix compounds and carriers.

14. The method according to claim 13, wherein the matrix compounds and carriers are selected from the group of type I and/or type III and/or type IV collagens, scleroproteins, gelatines, chitosans, glucosamines, glycosaminoglycans (GAGs), heparin sulfate proteoglycans, sulfated glycoproteins, growth factors, crosslinked polysaccharides, crosslinked polypeptides, and mixtures thereof.

15. The method according to claim 1, wherein the at least one three-dimensional sweat gland equivalent provided in method step a) comprises at least one cell type, selected from the group of (i) clear cells, dark cells, and myoepithelial cells, (ii) duct cells, and (iii) mixtures thereof.
